(19) United States
(12) Patent Application Publication
(10) Pub. No.: US 2013/0136704 A1
(43) Pub. Date: May 30, 2013

(54) P-MENTHANE-3,8-DIOL ISOMER MIXTURE, COMPOSITION COMPRISING THE SAME, AND PRODUCTS COMPRISING THE MIXTURE OR COMPOSITION

(71) Applicant: TAKASAGO INTERNATIONAL CORPORATION, Tokyo (JP)

(72) Inventors: Yasuhiro KOMATSUKI, Kamakura-shi (JP); Kenya ISHIDA, Yokohama-shi (JP); Kenichiro SHIROYAMA, Odawara-shi (JP)

(73) Assignee: TAKASAGO INTERNATIONAL CORPORATION, Tokyo (JP)

(21) Appl. No.: 13/686,655

(22) Filed: Nov. 27, 2012

(30) Foreign Application Priority Data
Nov. 28, 2011 (JP) 2011-259259

(51) Int. Cl. A61K 8/34; A61Q 11/00; A61K 45/06; A61K 31/047; A01N 31/06

(52) U.S. Cl. CPC A61K 8/345; A61K 31/047; A01N 31/06; A61K 45/06; A61Q 11/00
USPC 424/49; 514/729; 514/692; 514/617; 514/551; 424/747; 514/613; 514/546; 424/742; 424/769; 424/756

(57) ABSTRACT

An object of the present invention is to provide a novel technology for preventing crystal precipitation of p-menthane-3,8-diol, and stable products utilizing this technology. Provided is a p-menthane-3,8-diol isomer mixture, wherein the mass ratio of cis isomers:trans isomers in terms of the relative configuration at Positions 3 and 4 is 55:45 to 45:55. Also provided are stable products and the like containing the p-menthane-3,8-diol isomer mixture.

### TECHNICAL FIELD

The present invention relates to a p-menthane-3,8-diol isomer mixture capable of remaining in a liquid state even at relatively low temperature. Moreover, the present invention relates to a composition, an insect pest repellent composition, and a sensory stimulant composition which contain the p-menthane-3,8-diol isomer mixture, as well as products containing the isomer mixture or any of the compositions.

### BACKGROUND ART

Conventionally, cooling sensation agents which provide a refreshing feeling (refreshing sensation) and a cool feeling (cooling sensation), namely a cooling sensation effect, to the skin, oral cavity, nose, or throat of a person have been used for tooth-cleaning agents, confectionery products (for example, chewing gums, candies, and the like), tobaccos, cataplasms, cosmetics, and the like. Currently, p-menthane-3,8-diol has been widely used as a substance which provides the refreshing sensation or the cooling sensation. Moreover, p-menthane-3,8-diol has been widely used also as an insect pest repellent for protection of the human body from insect pests such as mosquitoes, black flies, tropical rat mites, stable flies, ants, bed bugs, and the like. It is known that p-menthane-3,8-diol is produced by a ring-closure and hydration of citronellal (Patent Literature 1). In addition, it is known that p-menthane-3,8-diol produced from d-citronellal or 1-citronellal exists in a liquid or semi-solid state. Moreover, it is also widely known that p-menthane-3,8-diol can be produced by a hydration of isopulegol in a known method, and it is also known that (+)-cis-p-menthane-3,8-diol or (−)-trans-p-menthane-3,8-diol obtained from n-isopulegol is crystalline with a high melting point of 70° C. or above. Further, it is also known that when a solution of p-menthane-3,8-diol prepared has a concentration of p-menthane-3,8-diol of 60% or higher, the p-menthane-3,8-diol is in a supersaturation state, and crystals thereof precipitate (Patent Literature 2).

Here, p-menthane-3,8-diol in a liquid state is easy to use as a cooling sensation agent or the like with a desired good handleability. Hence, methods for preventing crystal precipitation have been studied. Known technologies for preventing the crystal precipitation of p-menthane-3,8-diol include a method for preventing solidification by preparing a mixture of p-menthane-3,8-diol-citronellal acetal and isopulegol (Patent Literature 2), and a method in which a microemulsion is formed by using isopropanol (Non-Patent Literature 1).

### CITATION LIST

### Patent Literature

[Patent Literature 1] U.S. Pat. No. 6,328,982 B1
[Patent Literature 2] US 2010/0278755 A1

### Non-Patent Literature

[Non-Patent Literature 1] Chemistry & Biodiversity Volume 6, Issue 6, 934-947, (2009)

### SUMMARY OF INVENTION

### Technical Problems

However, in the case of each of the above-described methods, the characteristic odor of isopulegol or isopropanol is present. In addition, citronellal is formed with the elapse of time because of hydrolysis of p-menthane-3,8-diol-citronellal acetal, and the citronellal may increase the odor. Hence, when p-menthane-3,8-diol is blended with foods, cosmetics, or other products by using these formulation technologies, the amount of p-menthane-3,8-diol blended may be limited, and even the application of p-menthane-3,8-diol may also be limited.

Moreover, there is no technology for preventing the crystal precipitation of p-menthane-3,8-diol other than these methods. Further, there is no technology for controlling the crystal precipitation by freely controlling the ratio of 8 isomers of p-menthane-3,8-diol.

Accordingly, an object of the present invention is to lower the melting point of a p-menthane-3,8-diol isomer mixture, thereby prevent solidification and keep a liquid state, and also to provide products on which the improvement effect is reflected. More specifically, an object of the present invention is to provide a p-menthane-3,8-diol isomer mixture which stably remains liquid during production or in products, without impairing a cooling sensation effect, a repellent effect, and the like, as well as to various products with which the p-menthane-3,8-diol isomer mixture is blended.

### Solution to Problems

To solve the above-described problems, the present inventors have made earnest studies. As a result, the present inventors have found that, when the weight ratio of cis isomers:trans isomers in terms of the relative configuration at Positions 3 and 4 is 55:45 to 45:55 in a p-menthane-3,8-diol isomer mixture represented by the following chemical formula (I), the p-menthane-3,8-diol isomer mixture remains liquid even under a low-temperature situation. This finding has led to the completion of the present invention.

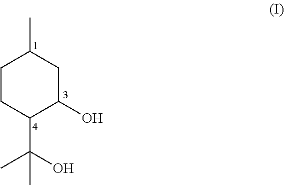

Specifically, the present invention relates to the following items [1] to [10].

[1] A p-menthane-3,8-diol isomer mixture, wherein

the weight ratio of cis isomers:trans isomers in terms of the relative configuration at Positions 3 and 4 is 55:45 to 45:55.

[2] A composition obtained by mixing the p-menthane-3,8-diol isomer mixture according to item [1] with an organic solvent.

[3] The composition according to item [2], wherein

the organic solvent is one or more selected from monohydroxy alcohols, polyols, ester oils, hydrocarbons, and silicone oils.

[4] The composition according to item [2] or [3], wherein

the weight ratio of the p-menthane-3,8-diol isomer mixture:the organic solvent is 90:10 to 30:70.

[5] The composition according to any one of items [2] to [4], wherein

the organic solvent is a monohydroxy alcohol or polyol having 2 to 20 carbon atoms.

[6] An insect pest repellent composition, comprising the p-menthane-3,8-diol isomer mixture according to item [1].

[7] The insect pest repellent composition according to item [6], further comprising at least one component selected from N,N-diethyl-m-toluamide (DEET), 3-(N-n-butyl-N-acetyl)aminopropionic acid ethyl ester, and 1-methylpropyl 2-(2-hydroxyethyl)-1-piperidinecarboxylate.

[8] A sensory stimulant composition, comprising the p-menthane-3,8-diol isomer mixture according to item [1] in combination with at least one component selected from menthol, isopulegol, menthone, camphor, pulegol, cineol, mint oil, 3-menthoxypropane-1,2-diol, N-alkyl-p-menthane-3-carboxamide, 3-menthoxy-2-methylpropane-1,2-diol, 2-(menthoxy)ethanol, 2-methyl-3-(1-menthoxy)propane-1,2-diol, 3-menthoxypropan-1-ol, 4-1-menthoxybutan-1-ol, menthyl 3-hydroxybutanoate, 1-(2-hydroxy-4-methylcyclohexyl)ethanone, menthyl lactate, menthol glycerin ketal, N-methyl-2,2-isopropylmethyl-3-methylbutanamide, menthyl glyoxylate, menthyl succinate, menthyl glutarate, peppermint oil, spearmint oil, eucalyptus oil, vanillyl ethyl ether, vanillyl propyl ether, vanillin propylene glycol acetal, ethylvanillin propylene glycol acetal, capsaicin, gingerol, vanillyl butyl ether, 4-(menthoxymethyl)-2-(3′-methoxy-4′-hydroxyphenyl)-1,3-diox olane, capsicum pepper oil, capsicum pepper oleoresin, ginger oleoresin, nonylic acid vanillyl amide, jambu oleoresin, Japanese pepper extract, sanshool-I, sanshool-II, sanshoamide, black pepper extract, chavicine, piperine, and spilanthol.

[9] A cosmetic, daily use product, oral cavity composition, or pharmaceutical, comprising

the p-menthane-3,8-diol isomer mixture according to item [1] or the composition according to any one of items [2] to [8], wherein

the content of the p-menthane-3,8-diol isomer mixture is 0.01 to 30% by weight.

[10] A method for preventing crystallization of a p-menthane-3,8-diol isomer mixture, comprising

mixing cis isomers and trans isomers in terms of the relative configuration at Positions 3 and 4 with each other at such an ratio that the cis isomers account for 55 to 45% by weight, whereas the trans isomers account for 45 to 55% by weight, relative to the total content of the p-menthane-3,8-diol isomer mixture.

### Advantageous Effects of Invention

In the present invention, the weight ratio of cis isomers:trans isomers in terms of the relative configuration at Positions 3 and 4 in the p-menthane-3,8-diol isomer mixture is adjusted to 55:45 to 45:55. Hence, the present invention makes it possible to provide a p-menthane-3,8-diol isomer mixture which can be blended in the liquid state with various formulations, without impairing effects as known or well-known cooling sensation agents, repellents, and the like, and without solidification during production or in products. Moreover, the present invention also makes it possible to obtain a sensory stimulant composition and an insect pest repellent composition which have a desired good handleability. Further, the above-described effects can be achieved similarly in the forms of products comprising the isomer mixture of the present invention, and it is also possible to obtain products, such as cosmetics, toiletry products, bath additives, and pharmaceuticals, which have desired effects as a sensory stimulant composition, an insect pest repellent composition, or the like, and which are in stable forms.

### DESCRIPTION OF EMBODIMENTS

Some methods for synthesizing p-menthane-3,8-diol represented by the above-described formula (I) have been reported so far. For example, Patent Literature 1 mentioned above reports a method for producing p-menthane-3,8-diol by a reaction of citronellal with a 0.02 to 1.0% by weight aqueous sulfuric acid solution.

It is known that p-menthane-3,8-diol has a nature of being very susceptible to crystal precipitation as described above, and that p-menthane-3,8-diol produced from d-citronellal or 1-citronellal exists in a semi-solid state. Moreover, it is widely known that p-menthane-3,8-diol can be produced by a hydration of isopulegol in a known method, and it is known that (+)-cis-p-menthane-3,8-diol or (−)-trans-p-menthane-3,8-diol obtained from n-isopulegol is crystalline with a high melting point of 70° C. or above. Further, it is also known that when the concentration of p-menthane-3,8-diol is 60% or higher, the p-menthane-3,8-diol is in a supersaturation state, and crystals thereof precipitate (see Patent Literature 2 mentioned above).

In addition, p-menthane-3,8-diol has three asymmetric carbons, and hence has 8 stereoisomers. There has been no report so far on a case where change in the solidification-susceptible nature is examined by changing the ratio of these isomers freely.

As a result of our earnest studies, the present inventors have found that a p-menthane-3,8-diol isomer mixture, wherein the weight ratio of cis isomers:trans isomers in terms of the relative configuration at Positions 3 and 4 is 55:45 to 45:55, astonishingly does not solidify, but remains liquid for a long period (for example, 3 days or longer) even under low-temperature situations (for example, 5° C. or below, even −20° C. or below), without impairing a cooling sensation effect or a repellent effect.

Based on this finding, the present inventors have completed a p-menthane-3,8-diol isomer mixture of the present invention, a composition comprising this isomer mixture, an insect pest repellent composition comprising this isomer mixture, a sensory stimulant composition comprising this isomer mixture, and products comprising any of these.

Hereinafter, completed embodiments of the present invention will be described in detail.

<p-Menthane-3,8-Diol Isomer Mixture>

In the case of the p-menthane-3,8-diol isomer mixture (hereinafter, also referred to simply as “isomer mixture”) of the present invention, it is only necessary to pay attention to the structure at Positions 3 and 4 of the p-menthane-3,8-diol contained. It is only necessary that the weight ratio of cis isomers:trans isomers in terms of the relative configuration at Positions 3 and 4 be 55:45 to 45:55, and preferably 53:47 to 47:53. Moreover, the p-menthane-3,8-diol contained in the isomer mixture may be S isomers or R isomers in terms of the structure at Position 1. Hence, for example, when the isomer mixture contains a large amount of the (1R, 3S, 4R) isomer (a cis isomer in terms of the relative configuration at Positions 3 and 4), and a small amount of the (1R, 3R, 4R) isomer (a trans isomer in terms of the relative configuration at Positions 3 and 4), it is possible to adjust the weight ratio of cis isomers:trans isomers in terms of the relative configuration at Positions 3 and 4 to 55:45 to 45:55, by adding the (1S, 3S, 4S) isomer (a trans isomer in terms of the relative configuration at Positions 3 and 4) to the isomer mixture. Note, however, that the structure at Position 1 of the p-menthane-3,8-diol isomers contained in the isomer mixture is desirably such that the weight ratio of (1R) isomers:(1S) isomers is 10:90 to 90:10, and preferably 70:30 to 30:70, for a reason that the lower the optical purity of citronellal, which is used as a raw material for the production, the lower the production cost is, and the more economical the production is, and other reasons.

The method for producing the isomer mixture is not particularly limited, and, for example, a method known to those skilled in the art can be employed in which any one of d-citronellal, l-citronellal, and dl-citronellal is used as a raw material. Whichever citronellal is selected as the raw material, a isomer mixture obtainable by this known method is such that cis isomers, namely (3S, 4R) isomers or (3R, 4S) isomers, account for 60 to 70% by weight, whereas trans isomers, namely (3R, 4R) isomers or (3S, 4S) isomers, accounts for 30 to 40% by weight, relative to the entire composition of the isomer mixture. The isomer mixture according to the present invention can be produced by adding an appropriate amount of an isomer mixture (the trans isomers in terms of the relative configuration at Positions 3 and 4 account for almost 100% by weight) obtained by a hydration of normal isopulegol or isoisopulegol to the isomer mixture having the above constitution. Moreover, the isomer mixture according to the present invention can also be produced in a one-pot manner by employing a system in which citronellal and an appropriate amount of normal isopulegol or isoisopulegol are coexistent, and by deriving an isomer mixture from the citronellal, and then deriving an isomer mixture (the trans isomers in terms of the relative configuration at Positions 3 and 4 account for almost 100% by mass) from the n-isopulegol or iso-isopulegol.

Moreover, the isomer mixture according to the present invention can also be obtained as follows. Specifically, by ring closure of citronellal with an appropriate acid catalyst, an mixture of isopulegol isomers is produced in which the weight ratio of cis isomers:trans isomers in terms of the relative configuration at positions 3 and 4 is 55:45 to 45:55. Then, the isomer mixture is obtained by a hydration of the obtained mixture of isopulegol isomers.

Moreover, when the isomer mixture of the present invention alone is contained in a product to be described later, or the composition, the insect pest repellent composition, the sensory stimulant composition, or the like to be described later is contained in a product, it is necessary to change, as appropriate, the range and method of application, depending on the kind, purpose of use, and the like of the product. In general, the isomer mixture of the present invention is preferably used at a concentration of 0.0001 to 90% by weight, and particularly 0.01 to 30% by weight, relative to the entire composition of the product.

<Composition>

The present invention also relates to a composition obtained by mixing the isomer mixture with an organic solvent. The organic solvent can be selected as appropriate, depending on the use of the composition. The composition may be used, for example, a tooth-cleaning agent, confectionery product (for example, a chewing gum, a candy, or the like), beverage, tobacco, cataplasm, shampoo, cosmetic, or the like having a cooling sensation effect; a cream, lotion, spray, or the like having an insect pest repellent effect; or the like. When mixed with an organic solvent, the isomer mixture of the present invention may serve as a composition which undergoes less crystal precipitate and which has a better handlability.

In the present invention, the kind of the organic solvent is not particularly limited, and examples thereof include lower alcohols such as ethanol, 1-propanol, 2-propanol, 2,2-dimethyl-1-propanol, 2-methyl-1-propanol, 1-butanol, 2-butanol, and tert-butanol; higher alcohols such as 2-hexyldecanol, oleyl alcohol, 2-octyldodecanol, batyl alcohol, cetanol, and stearyl alcohol; polyols such as glycerin, diglycerin, polyglycerin, ethylene glycol, diethylene glycol, triethylene glycol, polyethylene glycol, propylene glycol, dipropylene glycol, isoprene glycol, 3-methyl-1,3-butanediol, 1,3-butanediol, sorbitol, and maltitol; hydrocarbons such as squalane, liquid paraffin, liquid lanolin, vaseline, and solid paraffin; esters such as isopropyl palmitate, butyl stearate, isopropyl myristate, diethyl phthalate, myristyl lactate, diisopropyl adipate, cetyl myristate, cetyl lactate, 1-isostearoyl-3-myristoylglycerol, cholesteryl isostearate, cetyl 2-ethylhexanoate, 2-ethylhexyl palmitate, 2-octyldodecyl myristate, neopentylglycol di-2-ethylhexanoate, 2-octyldodecyl oleate, glycerol triisostearate, dicaprylyl carbonate, and glyceryl di-para-methoxycinnamate mono-2-ethylhexanoate; glycol ethers such as ethylene glycol monomethyl ether, ethylene glycol monoethyl ether, ethylene glycol monopropyl ether, diethylene glycol monomethyl ether, diethylene glycol monoethyl ether, diethylene glycol monopropyl ether, propylene glycol monoethyl ether, propylene glycol monopropyl ether, dipropylene glycol monoethyl ether, and dipropylene glycol monopropyl ether; fats and fatty oils such as safflower oil, sunflower oil, rosemary oil, jojoba oil, Macadamia nut oil, olive oil, camellia oil, castor oil, orange oil, rice bran oil, triethylhexanoin, and glyceryl tri(caprylate/caprate); silicone oils including linear chain polysiloxanes such as dimethyl polysiloxane, methyl phenyl polysiloxane, and diphenyl polysiloxane, cyclic polysiloxanes such as octamethylcyclotetrasiloxane, decamethylcyclopentasiloxane, and dodecamethylcyclohexasiloxane, modified polysiloxanes such as amino-modified polysiloxanes, polyether-modified polysiloxanes, alkyl-modified polysiloxanes, and fluorine-modified polysiloxanes, and the like; and the like.

However, the organic solvent is preferably one or more selected from monohydroxy alcohols, polyols, ester oils, hydrocarbons, and silicone oils, more preferably a monohydroxy alcohol or polyol having 2 to 20 carbon atoms, and particularly preferably at least one of ethanol, hexyldecanol, propylene glycol, triethylhexanoin, dipropylene glycol diethyl ether, and triethylene glycol, because of a high effect of preventing crystal precipitation.

Moreover, the weight ratio of the isomer mixture:the organic solvent in the composition is preferably 90:10 to 30:70, and particularly preferably 80:20 to 50:50. This is because, if the weight ratio of the isomer mixture:the organic solvent is higher than 90:lower than 10, such a problem may rarely occur that a quite trace amount of crystals are formed when the isomer mixture is stored for a long period at a low temperature or is subjected to an impact. This is also because, if the weight ratio is lower than 30:higher than 70, such a problem may occur that a desired effect of cooling sensation, repellence, or the like is impaired.

<Insect Pest Repellent Composition>

The present invention also relates to an insect pest repellent composition comprising the isomer mixture. Here, the insect pest repellent composition of the present invention, for example, means a composition for repelling insect pests such as mosquitoes, black flies, tropical rat mites, stable flies, ants, bed bugs, fleas, and horse-flies. The isomer mixture is preferably used as an insect pest repellent composition, because the isomer mixture has a high repellent effect against insect pests, in particular, haematophagous insect pests such as mosquitoes and black flies, and is a naturally occurring, low odor, and highly safe component derived from lemon eucalyptus. The insect pest repellent composition according to the present invention is liquid, and contains no solid contents, or contains a small amount of solid contents, if solid contents are contained. Hence, this insect pest repellent composition is preferably used in the forms of emulsions, oil solutions, wettable powders, sprays, liniments, powders, granules, capsules, or sheets.

The insect pest repellent composition of the present invention contains the isomer mixture of the present invention in an amount of preferably 0.01 to 90% by weight, and particularly preferably 0.1 to 30% by weight. This is because, if the content of the isomer mixture in the insect pest repellent composition is less than 0.01% by weight, for example, there is a possibility that a desired sufficient repellent effect is not exhibited. This is also because, if the content exceeds 90% by weight, stickiness to the skin or an excessive cooling sensation effect may cause an unpleasant sensation.

In the repellent composition, the isomer mixture may be used in combination with any of N,N-diethyl-m-toluamide, 1-methylpropyl 2-(2-hydroxyethyl)-1-piperidinecarboxylate, 2,3,4,5-bis(Δ2-butylene)tetrahydrofurfural, 3,4-caranediol, di-n-propyl isocinchoronate, 3-(N-n-butyl-N-acetyl)aminopropionic acid ethyl ester di-n-butyl succinate, 2-ethyl-1,3-hexanediol, 2-hydroxyoctyl sulfide, dimethyl phthalate, and (N-carbo-sec-butyloxy)-2-(2′-hydroxyethyl)piperidine, as well as any of plant essential oils such as citronella oil, lemongrass oil, hyssop oil, horseradish oil, bay oil, Angelica dahurica oil, fennel oil, basil oil, star anise oil, calamus oil, oregano oil, sandalwood oil, yarrow oil, perilla oil, valerian oil, ginger oil, palmarosa oil, laurel oil, ylang-ylang oil and clove oil, or the like.

Moreover, the insect pest repellent composition of the present invention preferably contains at least one component selected from N,N-diethyl-m-toluamide (DEET), 3-(N-n-butyl-N-acetyl)aminopropionic acid ethyl ester, and 1-methylpropyl 2-(2-hydroxyethyl)-1-piperidinecarboxylate. This is because these components have conventionally been used as components in insect pest repellents, and because a synergistic effect of insect pest repellence can be obtained, when any of these components is used together with the isomer mixture of the present invention. The insect pest repellent composition preferably contains any of these components in an amount of 0.1 to 50% by weight, in particular, 1 to 25% by weight relative to the isomer mixture of the present invention.

<Sensory Stimulant Composition>

The present invention also relates to a sensory stimulant composition comprising the isomer mixture.

Here, the sensory stimulant composition of the present invention means a composition which provides an effect of stimulating a sense, and the effect of stimulating a sense includes a cooling sensation effect and a warming sensation. Hence, the sensory stimulant composition of the present invention can be used as a superordinate concept encompassing a cooling sensation composition which provides a cooling sensation effect to the skin, oral cavity, nose, or throat of a person, and a warming sensation composition which provides a warming sensation effect to the skin, oral cavity, nose, or throat of a person.

Moreover, the sensory stimulant composition of the present invention can be blended as appropriate with a flavor and/or fragrance composition, a food or beverage, a cosmetic, a daily use product, an oral cavity composition, or a pharmaceutical.

When the sensory stimulant composition is prepared, the amount of the isomer mixture blended is preferably such that the isomer mixture is used at a concentration of 0.0001 to 20% by weight, in particular, 0.001 to 5% by weight, relative to the entire composition of the sensory stimulant composition, in general. However, it is necessary to change, as appropriate, the range and method of application, depending on the application of the sensory stimulant composition, the kind, purpose of use, and the like of the product in which the sensory stimulant composition is contained, and the like.

<<Cooling Sensation Composition>>

The isomer mixture of the present invention can serve as a cooling sensation component, and has a strong and long-lasting cooling sensation effect. Hence, a cooling sensation composition having a cooling sensation effect can be prepared by adding this isomer mixture.

Moreover, in addition to the isomer mixture, various components may be mixed with the cooling sensation composition containing the isomer mixture depending on the purpose of use. For example, a cooling sensation composition having an enhanced cooling sensation strength can be prepared by, for example, using, in combination, at least one cooling sensation component selected from menthol, isopulegol, menthone, camphor, pulegol, cineol, mint oil, 3-menthoxypropane-1,2-diol, N-alkyl-p-menthane-3-carboxamide, 3-menthoxy-2-methylpropane-1,2-diol, 2-(menthoxy)ethanol, 2-methyl-3-(1-menthoxy)propane-1,2-diol, 3-menthoxypropan-1-ol, 4-1-menthoxybutan-1-ol, menthyl 3-hydroxybutanoate, 1-(2-hydroxy-4-methylcyclohexyl)-ethanone, menthyl lactate, menthol glycerin ketal, N-methyl-2,2-isopropylmethyl-3-methylbutaneamide, menthyl glyoxylate, menthyl succinate, menthyl glutarate, peppermint oil, spearmint oil, and eucalyptus oil, as the cooling sensation component other than p-menthane-3,8-diol. Moreover, menthol, isopulegol, 3-menthoxypropane-1,2-diol, 2-(menthoxy)ethanol, or menthyl 3-hydroxybutanoate is particularly preferably used as the cooling sensation component other than p-menthane-3,8-diol, because a desired cooling sensation effect can be provided freely.

In the cooling sensation composition of the present invention, the isomer mixture and the cooling sensation components other than p-menthane-3,8-diol may be used at any ratio, as long as the effects of the present invention are not impaired. The mass ratio of the p-menthane-3,8-diol isomer composition:other cooling sensation components is preferably in a range from 1:99 to 95:5.

Moreover, since the cooling sensation composition is intended for a cooling sensation effect, no warming sensation component is contained, or the content by weight of the warming sensation component in the cooling sensation composition is generally set to 0.001 to 0.95 times, preferably 0.003 to 0.5 times the total weight of the isomer mixture and the cooling sensation components, so that the content can be within a range where the warming sensation component does not provide a warming sensation effect. In this case, the addition of the warming sensation component to the cooling sensation composition at the above-described ratio in the sensory stimulant composition of the present invention results in further improvement in the cooling sensation effect, and enhancement in the cooling sensation effect.

<<Warming Sensation Composition>>

A warming sensation composition comprising the isomer mixture is produced by mixing the isomer mixture with various warming sensation components selected depending on the purpose. For example, a warming sensation composition having an enhanced warming sensation strength can be prepared by using, as a component other than p-menthane-3,8-diol, at least one warming sensation component selected from vanillyl ethyl ether, vanillyl propyl ether, vanillin propylene glycol acetal, ethylvanillin propylene glycol acetal, capsaicin, gingerol, vanillyl butyl ether, 4-(menthoxymethyl)-2-(3′-methoxy-4′-hydroxyphenyl)-1,3-diox olane, capsicum pepper oil, capsicum pepper oleoresin, ginger oleoresin, nonylic acid vanillyl amide, jambu oleoresin, Japanese pepper extract, sanshool-I, sanshool-II, sanshoamide, black pepper extract, chavicine, piperine, and spilanthol, in combination with the isomer mixture. Moreover, vanillyl ethyl ether, capsaicin, gingerol, vanillyl butyl ether, capsicum pepper oil, or spilanthol is particularly preferably used as the warming sensation component, because a desired warming sensation effect can be provided freely.

In the warming sensation composition of the present invention, the isomer mixture and the above-described warming sensation component can be used at any ratio, as long as the warming sensation effect is not impaired. The mass ratio of the p-menthane-3,8-diol isomer composition:the warming sensation component is preferably in a range of 0.1:99.9 to 10:90.

Moreover, since the warming sensation composition is intended for a warming sensation effect, no cooling sensation components other than p-menthane-3,8-diol are contained, or the content by weight of the cooling sensation components in the warming sensation composition is generally set to 0.001 to 0.95 times, and preferably 0.01 to 0.5 times the total weight of the warming sensation component, so that the content can be within a range where the cooling sensation components do not provide a cooling sensation effect.

<Product>

The present invention also relates to a product comprising the isomer mixture, composition, insect pest repellent composition, or sensory stimulant composition.

In the present invention, the above-described composition, insect pest repellent composition, or sensory stimulant composition can be blended directly with various products such as flavor and/or fragrance compositions, foods or beverages, cosmetics, daily use products, oral cavity compositions, and pharmaceuticals. Meanwhile, regarding the sensory stimulant composition, it is also possible to separately add the isomer mixture, the other cooling sensation components, and the warming sensation component to the product, and then mix them together.

<<Flavor and/or Fragrance Composition>>

A flavor and/or fragrance composition containing the isomer mixture, composition, insect pest repellent composition, or sensory stimulant composition, in particular, containing the sensory stimulant composition (hereinafter, also referred to simply as “flavor and/or fragrance composition”) may also contain flavor and/or fragrance components such as synthetic flavors and/or fragrances, essential oils, citrus oils, and the like. For example, a wide variety of flavor and/or fragrance components such as those described in “Collection of Well-known Prior Arts (flavors and fragrances), Part I” (issued by Japan Patent Office on Jan. 29, 1999) can be used. Of these components, typical examples include α-pinene, limonene, neral, geranial, 2,5-dimethyl-4-hydroxy-3(2H)-furanone, vanillin, ethylvanillin, geraniol, nerol, citronellol, cis-3-hexenol, phenylethyl alcohol, styralyl acetate, eugenol, rose oxide, linalool, benzaldehyde, muscone, ethylene brassylate, (1R,6S)-2,2,6-Trimethyl-cyclohexanecarboxylic acid ethyl ester, and the like.

In particular, the content of the sensory stimulant composition in the flavor and/or fragrance composition according to the present invention can be adjusted depending on the kinds of flavors and/or fragrances and other components blended together, the purpose of use of the flavor and/or fragrance composition according to the present invention, and the like. For example, in a flavor and/or fragrance composition for a cosmetic, the content of the sensory stimulant composition is preferably 0.0001 to 90% by weight, more preferably 0.001 to 70% by weight, and particularly preferably 0.01 to 50% by weight, relative to the total mass of the flavor and/or fragrance composition, in general.

In other words, the flavor and/or fragrance composition contains the isomer mixture in an amount of preferably 0.0001 to 90% by weight, and further preferably 0.01 to 50% by weight.

Meanwhile, in a flavor and/or fragrance composition for a food or beverage, the content of the sensory stimulant composition is preferably 0.0001 to 90% by weight, and more preferably 0.01 to 50% by weight, relative to the total weight of the flavor and/or fragrance composition, in general. Further, the same content is preferable in the case of a flavor and/or fragrance composition for a daily use product, an oral cavity composition, or a pharmaceutical.

In other words, the flavor and/or fragrance composition contains the isomer mixture in an amount of preferably 0.0001 to 90% by weight, and further preferably 0.01 to 50% by weight.

If necessary, the flavor and/or fragrance composition according to the present invention may contain one or more flavor- and/or fragrance-retaining agents generally used for ordinary flavor and/or fragrance compositions. In this case, examples of the flavor- and/or fragrance-retaining agents include ethylene glycol, propylene glycol, dipropylene glycol, glycerin, hexylglycol, benzyl benzoate, triethyl citrate, diethyl phthalate, Hercolyn, medium-chain fatty acid triglycerides, medium-chain fatty acid diglycerides, and the like. The flavor and/or fragrance composition may contain one or more of these flavor- and/or fragrance-retaining agents.

<<Food or Beverage>>

Specific examples of the food or beverage comprising the isomer mixture, composition, sensory stimulant composition, or flavor and/or fragrance composition, which are capable of providing a sensory stimulation, include beverages such as fruit juice beverages, alcoholic fruit beverages, dairy beverages, carbonated beverages, refreshing beverages, and drink preparations; cold desserts such as ice creams, sherbets, and ice pops; desserts such as jellies and crème caramels; Western confectionery products such as cakes, cookies, chocolates, and chewing gums; Japanese confectionery products such as steamed bean-jam buns, Yokan (a thick jellied dessert made of red bean paste, agar, and sugar), and Uiro (a traditional Japanese steamed cake made of rice flour and sugar); jams; candies; breads; tea beverages and other favorite beverages such as green tea, oolong tea, black tea, persimmon leaf tea, chamomile tea, Sasa veitchii tea, mulberry leaf tea, Houttuynia cordata tea, pu-erh tea, mate tea, rooibos tea, gymnema tea, guava tea, coffee, and cocoa; soups such as Japanese style soups, Western style soups, and Chinese style soups; flavor seasonings; foods such as various instant beverages; various snack foods; and the like. However, the food or beverage is not limited to these examples.

<<Cosmetic and Daily Use Product>>

Examples of the cosmetic and daily use product, which may be scented by containing the isomer mixture, composition, insect pest repellent composition, sensory stimulant composition, or flavor and/or fragrance composition, include fragrance products, basic skin care cosmetics, make-up cosmetics, hair cosmetics, sunscreen cosmetics, medicated cosmetics, hair-care products, soaps, body cleaning agents, bath agents, detergents, finishing softeners, cleaning agents, kitchen detergents, bleaching agents, aerosol agents, deodorant and/or aromatic agents, other utensil products, and the like.

More specifically,

examples of the fragrance products include perfumes, Eau de Parfum, Eau de Toilette, Eau de Cologne, and the like;

examples of the basic skin care cosmetics include face wash creams, vanishing creams, cleansing creams, cold creams, massage creams, emulsions, lotions, cosmetic serums, beauty packs, make-up removers, and the like;

examples of the make-up cosmetics include foundations, loose face powders, pressed face powders, talcum powders, lipsticks, lip balms, cheek rouges, eyeliners, mascaras, eye shadows, eyebrow colors, eye packs, nail enamels, enamel removers, and the like;

examples of the hair cosmetics include pomades, brilliantines, set lotions, hair sticks, hair solids, hair oils, hair treatments, hair creams, hair tonics, hair liquids, hair sprays, bandolines, hair-growing agents, hair dyes, and the like;

examples of the sunscreen cosmetics include suntan products, sunscreen products, and the like;

examples of the medicated cosmetics include antiperspirant, after-shaving lotions and gels, permanent wave agent, medicated soaps, medicated shampoo, medicated skin cosmetics, and the like;

examples of the hair-care products include shampoos, rinses, two-in-one shampoos, conditioners, treatments, hair packs, and the like;

examples of the soaps include toilet soaps, bath soaps, perfume soaps, transparent soaps, synthetic soaps, and the like;

examples of the body cleaning agents include body soaps, body shampoos, hand soaps, and the like;

examples of the bath agents include bath additives (bath salts, bath tablets, bath liquids, and the like), foam baths (bubble baths and the like), bath oils (bath perfumes, bath capsules, and the like), milk baths, bath jellies, bath cubes, and the like;

examples of the detergents include heavy or light duty detergents, liquid detergents, laundry soaps, compact detergent, powder soaps, and the like;

examples of the finishing softeners include softeners, furniture cares, and the like;

examples of the cleaning agents include cleansers, house cleaning agents, toilet cleaners, bath cleaners, glass cleaners, mold removers, cleaners for drain pipes, and the like;

examples of the kitchen detergents include kitchen soaps, kitchen synthetic soap, dish detergents, and the like;

examples of the bleaching agents include oxidation bleaching agents (chlorine-based bleaching agents, oxygen-based bleaching agents, and the like), reduction bleaching agent (sulfur-based bleaching agents, and the like), optical bleaching agents, and the like;

examples of the aerosol agents include spray-type aerosol agents, powder sprays, and the like;

examples of the deodorant and/or aromatic agents include solid-, gel- or liquid-type deodorant and/or aromatic agents, and the like; and

examples of the utensil products include tissue paper, toilet paper, and the like.

<<Oral Cavity Composition and Pharmaceutical>>

Examples of the oral cavity composition include tooth cleaning agents (toothpastes and tooth gels), oral cavity washes, mouth washes, troches, and the like.

Examples of the pharmaceutical include skin external preparations such as cataplasms and ointments, internal preparations, and the like.

<Form of Composition Used for Product>

Depending on the final form (for example, the form of the product such as liquid, solid, powder, gel, mist, or aerosol) of the product of the present invention, the isomer mixture, the composition obtained by mixing the isomer mixture with an organic solvent, the insect pest repellent composition, or the sensory stimulant composition may be added in various manners. Specifically, these may be added directly to the product. Alternatively, the isomer mixture alone or the composition containing the isomer mixture may be added in a liquid from, for example, after being dissolved in a monohydroxy alcohol or a polyol such as propylene glycol or glycerin. Further, the isomer mixture alone or the composition containing the isomer mixture may be added in a soluble form or a dispersed form, after being converted into a soluble form or being dispersed by emulsification by using a naturally occurring gum substance such as gum arabic or gum tragacanth, a surfactant (for example, a nonionic surfactant such as a glycerin fatty acid ester or a sucrose fatty acid ester, an anionic surfactant, an cationic surfactant, an amphoteric surfactant, or the like). Moreover, the isomer mixture alone or the composition containing the isomer mixture may be added in a powder form, after coating is formed by using an excipient such as a naturally occurring gum substance such as gum arabic, gelatin, dextrin, or the like, or may be added in a form of microcapsules after being treated with a capsule-forming agent. Moreover, the isomer mixture, the composition obtained by mixing the isomer mixture with an organic solvent, the insect pest repellent composition, or the sensory stimulant composition may be used in a stabilized and sustained-release form, after being included in an inclusion agent such as cyclodextrin.

In particular, the amount of the sensory stimulant composition added to the product for providing a cooling sensation or a sensory stimulation can be adjusted, depending on the kind and form of the product, effects and operations of providing a cooling sensation or sensory stimulation required by the product, and the like. In general, the amount of the sensory stimulant composition added is preferably 0.0001 to 90% by weight, and more preferably 0.01 to 20% by weight, in particular, relative to the weight of the product.

In other words, the product contains the isomer mixture in an amount of preferably 0.0001 to 90% by weight, and further preferably in an amount of 0.01 to 30% by weight.

### EXAMPLES

Hereinafter, the present invention is described more specifically based on Examples. However, the present invention is not limited to these examples at all. Any modifications may be made as appropriate within the scope of the present invention. Note that the following apparatus and device were used for identification and physical property measurement of products of Examples and test experiments.

Gas chromatograph: GC4000 (manufactured by GL Sciences Inc.)

Column: Rtx-1 (manufactured by SHIMADZU GLC Ltd.)

### Comparative Example 1

### Synthesis Example 1

Into a 4-necked 500-ml flask equipped with a stirrer, a thermometer, and a dropping funnel, 133.7 g of a 0.25% diluted sulfuric acid and 93 ml of toluene were introduced, and heated to 70° C. To this mixture, 77.1 g of dl-citronellal was added dropwise over 1 hour, and then a reaction was allowed to proceed at the same temperature for 9 hours. After cooling, the reaction liquid was neutralized by adding a 25% aqueous NaOH solution, and subjected to an extraction treatment with stirring by adding 150 ml of heptane. The phase-separated organic layer was washed with water, and then the solvent was removed by distillation, followed by vacuum distillation (100 to 120° C., 0.2 torr (26.7 Pa)). Thus, 64.2 g of a p-menthane-3,8-diol isomer composition was obtained. The mass ratio of cis isomers/trans isomers in terms of the relative configuration at Positions 3 and 4 was 62/38 in the obtained p-menthane-3,8-diol isomer mixture.

### Comparative Example 2

### Synthesis Example 2

To a 4-necked 100-ml flask equipped with a stirrer, a thermometer, and a dropping funnel, 18.1 g of 65% sulfuric acid and 30 g of heptane were added, and cooled with ice. To this mixture, 15.4 g of n-isopulegol was added dropwise over 1 hour, followed by stirring at the same temperature for 5 hours. The mixture was diluted by adding 30 g of water, and stirred for 30 minutes. Then, the organic layer was separated, and washed with a 5% aqueous NaOH solution, and then with water. The solvent was removed by distillation, heptane was added thereto, and crystals were precipitated at −20° C. The precipitates were filtered to obtain 16.9 g of a p-menthane-3,8-diol isomer composition. The mass ratio of cis isomers/trans isomers in terms of the relative configuration at Positions 3 and 4 was 0/100 in the obtained p-menthane-3,8-diol.

### Example 1

### p-Menthane-3,8-Diol Isomer Mixture

10.0 g of the p-menthane-3,8-diol isomer mixture obtained in (Synthesis Example 1) and 2.0 g of the p-menthane-3,8-diol obtained in (Synthesis Example 2) were completely dissolved in each other. The mass ratio of cis isomers/trans isomers in terms of the relative configuration at Positions 3 and 4 was 50/50 in the mixed p-menthane-3,8-diol isomer mixture.

### Example 2

### p-Menthane-3,8-Diol Isomer Mixture

Into a 4-necked 500-ml flask equipped with a stirrer, a thermometer, and a dropping funnel, 133.7 g of a 0.25% diluted sulfuric acid and 93 ml of toluene were introduced, and heated to 70° C. To this mixture, 77.1 g of dl-citronellal was added dropwise for 1 hour, and then a reaction was allowed to proceed at the same temperature for 9 hours. To this reaction liquid, 15.4 g of 1-isopulegol was added, and 12.0 g of concentrated sulfuric acid was added dropwise over 1 hour to the mixture under ice-cooling, followed by stirring at the same temperature for 3 hours. The reaction liquid was neutralized by adding a 25% aqueous NaOH solution, and was subjected to an extraction treatment with stirring by adding 150 ml of heptane. The phase-separated organic layer was washed with water, and then the solvent was removed by distillation, followed by vacuum distillation (100 to 120° C., 0.2 torr (26.7 Pa)). Thus, 64.2 g of a p-menthane-3,8-diol isomer composition was obtained. The mass ratio of cis isomers/trans isomers in terms of the relative configuration at Positions 3 and 4 was 52/48 in the obtained p-menthane-3,8-diol isomer mixture.

### Synthesis Example 3

To a 4-necked 500-ml flask equipped with a stirrer, a thermometer, a dropping funnel, and a reflux condenser, 77.1 g of dl-citronellal, 230 ml of toluene, and 1.42 g of titanium tetrachloride were added, and stirred at 130° C. for 6 hours. After cooling, 100 ml of a 1% aqueous NaOH solution was added to this reaction liquid, followed by phase separation. The solvent was removed by distillation from the organic layer, followed by vacuum distillation (80 to 100° C., 4.0 torr (533.3 Pa)). Thus, 45.2 g of an isopulegol isomer composition was obtained. The mass ratio of cis isomers/trans isomers in terms of the relative configuration at Positions 3 and 4 was 54/46 in the obtained isopulegol isomer mixture.

### Example 3

### p-Menthane-3,8-Diol Isomer Mixture

To a 4-necked 100-ml flask equipped with a stirrer, a thermometer, and a dropping funnel, 18.1 g of 65% sulfuric acid and 30 g of heptane were added, and cooled with ice. To this mixture, 15.4 g of the isopulegol isomer composition obtained in (Synthesis Example 3) was added dropwise over 1 hour, followed by stirring at the same temperature for 5 hours. After addition of 30 g of water, and stirring for 30 minutes, the organic layer was separated, and washed with a 5% aqueous NaOH solution and then with water, followed by vacuum distillation (100 to 120° C., 0.2 torr (26.7 Pa)). Thus, 12.3 g of a p-menthane-3,8-diol isomer composition was obtained. The mass ratio of cis isomers/trans isomers in terms of the relative configuration at Positions 3 and 4 was 53/47 in the obtained p-menthane-3,8-diol isomer mixture.

<<Test Example 1>>

The p-menthane-3,8-diol isomer mixtures obtained in Comparative Example 1 (Synthesis Example 1), Comparative Example 2 (Synthesis Example 2), Example 1, Example 2, and Example 3 were allowed to stand still at temperatures of 25° C., 5° C., or −20° C., and visually observed for their states. Tables 1 to 3 show the results.

In Tables 1 to 3, the signs ∘, Δ, and x indicate the following states, respectively.

∘: Completely dissolved

Δ: Partially solidified

x: Completely solidified

TABLE 1
25° C.

| | Comp. | Comp. | Example | Example | Example |
|---|---|---|---|---|---|
| | Ex. 1 | Ex. 2 | 1 | 2 | 3 |
| 1 hour later | ∘ | x | ∘ | ∘ | ∘ |
| 3 hours later | ∘ | x | ∘ | ∘ | ∘ |
| 8 hours later | Δ | x | ∘ | ∘ | ∘ |
| 24 hours later | x | x | ∘ | ∘ | ∘ |
| 48 hours later | x | x | ∘ | ∘ | ∘ |
| 72 hours later | x | x | ∘ | ∘ | ∘ |

TABLE 2
5° C.

| | Comp. | Comp. | Example | Example | Example |
|---|---|---|---|---|---|
| | Ex. 1 | Ex. 2 | 1 | 2 | 3 |
| 1 hour later | ∘ | x | ∘ | ∘ | ∘ |
| 3 hours later | Δ | x | ∘ | ∘ | ∘ |
| 8 hours later | x | x | ∘ | ∘ | ∘ |
| 24 hours later | x | x | ∘ | ∘ | ∘ |
| 48 hours later | x | x | ∘ | ∘ | ∘ |
| 72 hours later | x | x | ∘ | ∘ | ∘ |

TABLE 3
−20° C.

| | Comp. | Comp. | Example | Example | Example |
|---|---|---|---|---|---|
| | Ex. 1 | Ex. 2 | 1 | 2 | 3 |
| 1 hour later | Δ | x | ∘ | ∘ | ∘ |
| 3 hours later | x | x | ∘ | ∘ | ∘ |
| 8 hours later | x | x | ∘ | ∘ | ∘ |
| 24 hours later | x | x | ∘ | ∘ | ∘ |
| 48 hours later | x | x | ∘ | Δ | Δ |
| 72 hours later | x | x | ∘ | Δ | Δ |

It was found from Test Example 1 that the p-menthane-3,8-diol isomer mixtures of the present invention were stable for long periods at normal temperature and a low temperature (5° C.), and no crystals precipitated therein. Moreover, it was found that the p-menthane-3,8-diol isomer mixtures of the present invention were more stable than those of Comparative Examples even below the freezing point of water (even at −20° C.), and crystals were less likely to precipitate therein. From these results, it has been proved that the present invention makes it possible to provide a p-menthane-3,8-diol isomer mixture with a desired good handleability.

<<Test Example 2>>

The p-menthane-3,8-diol isomer mixtures obtained in Comparative Example 1, Comparative Example 2, Example 1, Example 2, and Example 3 were allowed to stand still at −40° C. for a long period, and completely solidified. Then, each solidified p-menthane-3,8-diol isomer mixture was allowed to stand still at a temperature of 50° C., and the time taken before complete dissolution was measured. Table 4 shows the results.

TABLE 4

| | Comp. | Comp. | Ex. | Ex. | Ex. |
|---|---|---|---|---|---|
| | Ex. 1 | Ex. 2 | 1 | 2 | 3 |
| Time before | 8.0 | Not | 1.0 | 1.5 | 1.5 |
| dissolution | (Crystals | dissolved | | | |
| (hours) | remained) | | | | |

From Test Example 2, it was found that the p-menthane-3,8-diol isomer mixtures of the present invention easily melted with mild and short-term heating, and had lower melting points than those of Comparative Examples. From those results, it has been proved that the present invention makes it possible to provide a p-menthane-3,8-diol isomer mixture with a desired good handleability.

### Comparative Example 3

The p-menthane-3,8-diol isomer mixture obtained in Comparative Example 1 was diluted with various solvents, and the following solutions of Formulation Examples were prepared (see Formulation Examples with odd numbers).

### Example 4

The p-menthane-3,8-diol isomer mixture obtained in Example 1 (hereinafter referred to as “PMD” in Example 4) was diluted with various solvents, and the following solutions (“compositions” according to the present application) of Formulation Examples were prepared (see Formulation Examples with even numbers).

Formulation Example 1: PMD of Comparative Example 1: 7 g, dicaprylyl carbonate: 3 g
Formulation Example 2: PMD of Example 1: 7 g, dicaprylyl carbonate: 3 g
Formulation Example 3: PMD of Comparative Example 1: 7 g, dipropylene glycol diethyl ether: 3 g
Formulation Example 4: PMD of Example 1: 7 g, dipropylene glycol diethyl ether: 3 g
Formulation Example 5: PMD of Comparative Example 1: 7 g, hexyldecanol: 3 g
Formulation Example 6: PMD of Example 1: 7 g, hexyldecanol: 3 g
Formulation Example 7: PMD of Comparative Example 1: 7 g, isoprene glycol: 3 g
Formulation Example 8: PMD of Example 1: 7 g, isoprene glycol:
3 g

Formulation Example 9: PMD of Comparative Example 1: 7 g, glycerin fatty acid ester: 3 g

Formulation Example 10: PMD of Example 1: 7 g, glycerin fatty acid ester: 3 g
Formulation Example 11: PMD of Comparative Example 1: 7 g, triethylhexanoin: 3 g

Formulation Example 12: PMD of Example 1: 7 g, triethylhexanoin: 3 g

Formulation Example 13: PMD of Comparative Example 1: 7 g, propylene glycol: 3 g
Formulation Example 14: PMD of Example 1: 7 g, propylene glycol: 3 g
Formulation Example 15: PMD of Comparative Example 1: 7 g, triethylene glycol: 3 g
Formulation Example 16: PMD of Example 1: 7 g, triethylene glycol: 3 g
Formulation Example 17: PMD of Comparative Example 1: 7 g, ethanol: 3 g
Formulation Example 18: PMD of Example 1: 7 g, ethanol: 3 g
Formulation Example 19: PMD of Comparative Example 1: 7 g, paraffin: 3 g
Formulation Example 20: PMD of Example 1: 7 g, paraffin: 3 g

<<Test Example 3>>

The above-described solutions of Formulation Examples were prepared, allowed to stand still at 25° C., 5° C., or −20° C., and observed for their states.

Tables 5 to 7 show the results of observation of the solutions of Formulation Examples at the temperatures.

In Tables 5 to 7, the signs ∘, Δ, and x indicate the following states, respectively.

∘: Completely dissolved

Δ: Partially solidified

x: Completely solidified

TABLE 5
25° C.

| | 3 | 8 | 24 | | |
|---|---|---|---|---|---|
| | hours | hours | hours | 3 days | 7 days |
| | later | later | later | later | later |
| Formulation Example 1 | ∘ | ∘ | ∘ | Δ | x |
| Formulation Example 2 | ∘ | ∘ | ∘ | ∘ | ∘ |
| Formulation Example 3 | ∘ | ∘ | ∘ | ∘ | Δ |
| Formulation Example 4 | ∘ | ∘ | ∘ | ∘ | ∘ |
| Formulation Example 5 | ∘ | ∘ | ∘ | ∘ | ∘ |
| Formulation Example 6 | ∘ | ∘ | ∘ | ∘ | ∘ |
| Formulation Example 7 | ∘ | ∘ | Δ | x | x |
| Formulation Example 8 | ∘ | ∘ | ∘ | ∘ | ∘ |
| Formulation Example 9 | ∘ | ∘ | ∘ | Δ | x |
| Formulation Example 10 | ∘ | ∘ | ∘ | ∘ | ∘ |
| Formulation Example 11 | ∘ | ∘ | ∘ | ∘ | ∘ |
| Formulation Example 12 | ∘ | ∘ | ∘ | ∘ | ∘ |
| Formulation Example 13 | ∘ | ∘ | Δ | x | x |
| Formulation Example 14 | ∘ | ∘ | ∘ | ∘ | ∘ |
| Formulation Example 15 | ∘ | ∘ | ∘ | ∘ | Δ |
| Formulation Example 16 | ∘ | ∘ | ∘ | ∘ | ∘ |
| Formulation Example 17 | ∘ | ∘ | ∘ | ∘ | ∘ |
| Formulation Example 18 | ∘ | ∘ | ∘ | ∘ | ∘ |
| Formulation Example 19 | ∘ | ∘ | Δ | x | x |
| Formulation Example 20 | ∘ | ∘ | ∘ | ∘ | ∘ |

TABLE 6
5° C.

| | 3 | 8 | 24 | | |
|---|---|---|---|---|---|
| | hours | hours | hours | 3 days | 7 days |
| | later | later | later | later | later |
| Formulation Example 1 | ∘ | ∘ | Δ | x | x |
| Formulation Example 2 | ∘ | ∘ | ∘ | ∘ | ∘ |
| Formulation Example 3 | ∘ | ∘ | Δ | x | x |
| Formulation Example 4 | ∘ | ∘ | ∘ | ∘ | ∘ |
| Formulation Example 5 | ∘ | ∘ | ∘ | ∘ | ∘ |
| Formulation Example 6 | ∘ | ∘ | ∘ | ∘ | ∘ |
| Formulation Example 7 | ∘ | ∘ | x | x | x |
| Formulation Example 8 | ∘ | ∘ | ∘ | ∘ | ∘ |
| Formulation Example 9 | ∘ | ∘ | Δ | x | x |
| Formulation Example 10 | ∘ | ∘ | ∘ | ∘ | ∘ |
| Formulation Example 11 | ∘ | ∘ | Δ | x | x |
| Formulation Example 12 | ∘ | ∘ | ∘ | ∘ | ∘ |
| Formulation Example 13 | ∘ | ∘ | Δ | x | x |
| Formulation Example 14 | ∘ | ∘ | ∘ | ∘ | ∘ |
| Formulation Example 15 | ∘ | ∘ | ∘ | Δ | x |
| Formulation Example 16 | ∘ | ∘ | ∘ | ∘ | ∘ |
| Formulation Example 17 | ∘ | ∘ | ∘ | ∘ | Δ |
| Formulation Example 18 | ∘ | ∘ | ∘ | ∘ | ∘ |
| Formulation Example 19 | ∘ | ∘ | Δ | x | x |
| Formulation Example 20 | ∘ | ∘ | ∘ | ∘ | ∘ |

TABLE 7
-20° C.

| | 3 | 8 | 24 | | |
|---|---|---|---|---|---|
| | hours | hours | hours | 3 days | 7 days |
| | later | later | later | later | later |
| Formulation Example 1 | Δ | x | x | x | x |
| Formulation Example 2 | ∘ | ∘ | ∘ | ∘ | Δ |
| Formulation Example 3 | ∘ | ∘ | Δ | x | x |
| Formulation Example 4 | ∘ | ∘ | ∘ | ∘ | ∘ |
| Formulation Example 5 | ∘ | ∘ | ∘ | Δ | x |
| Formulation Example 6 | ∘ | ∘ | ∘ | ∘ | ∘ |
| Formulation Example 7 | Δ | x | x | x | x |
| Formulation Example 8 | ∘ | ∘ | ∘ | ∘ | Δ |
| Formulation Example 9 | ∘ | Δ | x | x | x |
| Formulation Example 10 | ∘ | ∘ | ∘ | ∘ | Δ |
| Formulation Example 11 | ∘ | Δ | x | x | x |
| Formulation Example 12 | ∘ | ∘ | ∘ | ∘ | ∘ |
| Formulation Example 13 | Δ | x | x | x | x |
| Formulation Example 14 | ∘ | ∘ | ∘ | ∘ | ∘ |
| Formulation Example 15 | ∘ | ∘ | Δ | x | x |
| Formulation Example 16 | ∘ | ∘ | ∘ | ∘ | ∘ |
| Formulation Example 17 | ∘ | ∘ | ∘ | Δ | x |
| Formulation Example 18 | ∘ | ∘ | ∘ | ∘ | ∘ |
| Formulation Example 19 | Δ | x | Δ | x | x |
| Formulation Example 20 | ∘ | ∘ | ∘ | Δ | Δ |

From Tables 5 to 7, it has been found that it is preferable to use dicaprylyl carbonate, dipropylene glycol diethyl ether, hexyldecanol, isoprene glycol, glycerin fatty acid ester, triethylhexanoin, propylene glycol, triethylene glycol, ethanol, or paraffin as an organic solvent for the composition of the present invention, and it has been found that ethanol, hexyldecanol, propylene glycol, triethylhexanoin, dipropylene glycol diethyl ether, and triethylene glycol are particularly preferable for storage at −20° C.

### Example 5

### Repellent Lotion Against Harmful Insect Pests (Insect Pest Repellent Composition)

A repellent lotion against harmful insect pests was prepared according to the following formula.

<Formula of Repellent Lotion Against Harmful Insect Pests>

| (Component) | (Blended amount: g) |
|---|---|
| p-Menthane-3,8-diol isomer mixture of Example 1 | 10.00 |
| 99.5% Ethanol | 80.00 |
| Purified water | the balance |
| Total | 100.00 |

The repellent lotion against harmful insect pests prepared according to the above formula had a sufficient repellent or cooling sensation effect, and no crystals of p-menthane-3,8-diol precipitated therein even in a long-term storage at a low temperature.

### Example 6

### Toothpaste

A toothpaste was prepared according to the following formula.

<Formula of Toothpaste>

| (Component) | (Blended amount: g) |
|---|---|
| 1-Menthol | 0.25 |
| p-Menthane-3,8-diol isomer mixture of Example 1 | 0.05 |
| Calcium hydrogen phosphate (dihydrate) | 50.00 |
| Glycerin | 25.00 |
| Sodium lauryl sulfate | 1.40 |
| Carboxymethyl cellulose sodium | 1.50 |
| Sodium saccharin | 0.20 |
| Sodium benzoate | 0.10 |
| Strawberry-type flavor | 0.70 |
| (manufactured by Takasago International Corporation) | |
| Purified water | the balance |
| Total | 100.00 |

The toothpaste prepared according to the above formula had a sufficient cooling sensation effect, and no crystals of p-menthane-3,8-diol precipitated therein even in a long-term storage at a low temperature.

### Example 7

### Chewing Gum

A chewing gum was prepared according to the following formula.

<Formula of Chewing Gum>

| (Component) | (Blended amount: g) |
|---|---|
| 1-Menthol | 0.01 |
| p-Menthane-3,8-diol isomer mixture of Example 1 | 0.01 |
| Gum base | 24.00 |
| Corn syrup (42DE) | 6.70 |
| Glycerin | 1.10 |
| Sugar | 67.18 |
| Peppermint-type flavor | 1.00 |
| (manufactured by Takasago International Corporation) | |
| Total | 100.00 |

The chewing gum prepared according to the above formula had a sufficient cooling sensation effect, and no crystals of p-menthane-3,8-diol precipitated therein even in a long-term storage at a low temperature.

### Example 8

### Flavor and/or Fragrance Composition

A flavor and/or fragrance composition containing the p-menthane-3,8-diol isomer mixture of Example 1 was prepared in a usual manner according to the following formula (blended amounts are expressed in parts by mass).

<Formula of Flavor and/or Fragrance Composition>

| (Component) | (Blended amount: g) |
|---|---|
| Apple base | 8.0 |
| (manufactured by Takasago International | |
| Corporation) | |
| Bergamot oil | 14.0 |
| Ethyl acetoacetate | 5.0 |
| Methyl dihydrojasmonate | 23.0 |
| Laurinal | 3.0 |
| Levosandol | 4.0 |
| (manufactured by Takasago International | |
| Corporation) | |
| Orange oil | 8.0 |
| 10-Oxa-16-hexadecanolide | 8.0 |
| Phenoxanol (manufactured by IFF) | 6.0 |
| Styralyl acetate | 3.0 |
| Thesaron | 8.0 |
| (manufactured by Takasago International | |
| Corporation) | |
| p-Menthane-3,8-diol isomer mixture of Example 1 | 30.0 |

### Example 9

### Shampoo

According to the following formula, 100 g of a shampoo scented with the above-described flavor and/or fragrance composition of Example 8 at 1.0% was prepared. This shampoo had a sufficient cooling sensation effect, and no crystals of p-menthane-3,8-diol precipitated therein even in a long-term storage at a low temperature.

<Formula of Shampoo>

| (Component) | (Blended amount: g) |
|---|---|
| Polyoxyethylene lauryl ether sodium sulfate | 14.00 |
| Lauramidopropyl betaine | 4.00 |
| Coconut oil fatty acid diethanolamide | 3.00 |
| Cationized cellulose | 0.50 |
| Ethylene glycol distearate | 1.00 |
| Ethyl p-hydroxybenzoate | 0.25 |
| Citric acid | sufficient quantity |
| Flavor and/or fragrance composition of Example 8 | 1.00 |
| Purified water | the balance |
| Total | 100.00 |

### Example 10

### Sensory Stimulant Composition

A sensory stimulant composition containing the p-menthane-3,8-diol isomer mixture of Example 1 was prepared in a usual manner according to the following formula (blended amounts are expressed in parts by mass).

<Formula of Sensory Stimulant Composition>

| (Component) | (Blended amount: g) |
|---|---|
| p-Menthane-3,8-diol isomer mixture of Example 1 | 50.00 |
| 2-(Menthoxy) ethanol | 20.00 |
| Menthyl 3-hydroxybutanoate | 15.00 |
| 3-Menthoxypropane-1,2-diol | 10.00 |
| Isopulegol | 4.89 |
| Spilanthol | 0.10 |
| Vanillyl ethyl ether | 0.01 |
| Total | 100.00 |

### Example 11

### Chewing Gum

A chewing gum was prepared according to the following formula.

<Formula of Chewing Gum>

| (Component) | (Blended amount: g) |
|---|---|
| 1-Menthol | 0.01 |
| Sensory stimulant composition of Example 10 | 0.01 |
| Gum base | 24.00 |
| Corn syrup (42DE) | 6.70 |
| Glycerin | 1.10 |
| Sugar | 67.18 |
| Peppermint-type flavor | 1.00 |
| (manufactured by Takasago International Corporation) | |
| Total | 100.00 |

The chewing gum prepared according to the above-described formula had a cool and refreshing feel. It was noticed that the chewing gum of the above formula had a more emphasized refreshing feel and a more persistent cooling feel than a chewing gum using no sensory stimulant composition containing the p-menthane-3,8-diol isomer mixture of Example 1 of the present invention.

### INDUSTRIAL APPLICABILITY

The p-menthane-3,8-diol isomer mixture used in the present invention can serve as an excellent cooling sensation component or repellent component that are capable of remaining liquid even under a low-temperature situation without impairing a cooling sensation effect or repellent effect, as compared with p-menthane-3,8-diol isomer mixtures obtained by conventional production methods. Moreover, the present invention provides a sensory stimulant composition and an insect pest repellent composition which contain the isomer mixture, as well as a cosmetic, a daily use product, an oral cavity composition, and a pharmaceutical which contain the isomer mixture or the insect pest repellent composition.

1. A p-menthane-3,8-diol isomer mixture, wherein the mass ratio of cis isomers:trans isomers in terms of the relative configuration at Positions 3 and 4 is 55:45 to 45:55.

2. A composition obtained by mixing the p-menthane-3,8-diol isomer mixture according to claim 1 with an organic solvent.

3. The composition according to claim 2, wherein
the organic solvent is one or more selected from monoalcohols, polyols, ester oils, hydrocarbons, and silicone oils.

4. The composition according to claim 2, wherein
the mass ratio of the p-menthane-3,8-diol isomer mixture to the organic solvent is 90:10 to 30:70.

5. The composition according to claim 2, wherein
the organic solvent is a monoalcohol or polyol having 2 to 20 carbon atoms.

6. An insect pest repellent composition, comprising the p-menthane-3,8-diol isomer mixture according to claim 1.

7. The insect pest repellent composition according to claim 6, further comprising at least one component selected from N,N-diethyl-m-toluamide, 3-(N-n-butyl-N-acetyl)aminopropionic acid ethyl ester, and 1-methylpropyl 2-(2-hydroxyethyl)-1-piperidinecarboxylate.

8. A sensory stimulant composition, comprising the p-menthane-3,8-diol isomer mixture according to claim 1 in combination with at least one component selected from menthol, isopulegol, menthone, camphor, pulegol, cineol, mint oil, 3-menthoxypropane-1,2-diol, N-alkyl-p-menthane-3-carboxamide, 3-menthoxy-2-methylpropane-1,2-diol, 2-(menthoxy)ethanol, 2-methyl-3-(1-menthoxy)propane-1,2-diol, 3-menthoxypropan-1-ol, 4-1-menthoxybutan-1-ol, menthyl 3-hydroxybutanoate, 1-(2-hydroxy-4-methylcyclohexyl)ethanone, menthyl lactate, menthol glycerin ketal, N-methyl-2,2-isopropylmethyl-3-methylbutanamide, menthyl glyoxylate, menthyl succinate, menthyl glutarate, peppermint oil, spearmint oil, eucalyptus oil, vanillyl ethyl ether, vanillyl propyl ether, vanillin propylene glycol acetal, ethylvanillin propylene glycol acetal, capsaicin, gingerol, vanillyl butyl ether, 4-(menthoxymethyl)-2-(3′-methoxy-4′-hydroxyphenyl)-1,3-dioxolane, capsicum pepper oil, capsicum pepper oleoresin, ginger oleoresin, nonylic acid vanillyl amide, jambu oleoresin, Japanese pepper extract, sanshool-I, sanshool-II, sanshoamide, black pepper extract, chavicine, piperine, and spilanthol.

9. A cosmetic, daily use product, oral cavity composition, or pharmaceutical, comprising the p-menthane-3,8-diol isomer mixture according to claim 1, wherein
the content of the p-menthane-3,8-diol isomer mixture is 0.01 to 30% by weight.

10. A cosmetic, daily use product, oral cavity composition, or pharmaceutical, comprising the composition according to claim 2, wherein
the content of the p-menthane-3,8-diol isomer mixture is 0.01 to 30% by weight.

11. A cosmetic, daily use product, oral cavity composition, or pharmaceutical, comprising the composition according to claim 8, wherein
the content of the p-menthane-3,8-diol isomer mixture is 0.01 to 30% by weight.

12. A method for preventing crystallization of a p-menthane-3,8-diol isomer mixture, comprising
mixing cis isomers and trans isomers in terms of the relative configuration at Positions 3 and 4 with each other at such an ratio that the cis isomers account for 55 to 45% by weight, whereas the trans isomers account for 45 to 55% by mass, relative to the total content of the p-menthane-3,8-diol isomer mixture.
